(19) United States
(12) Patent Application Publication
(10) Pub. No.: US 2026/0151518 A1
(43) Pub. Date: Jun. 4, 2026

(54) LEUKOCYTE-SPECIFIC CELL PENETRATING MOLECULES

(71) Applicant: Amytrx Therapeutics, Inc., Nashville, TN (US)

(72) Inventors: Vernon Alvarez, Nashville, TN (US); Matthew A. Gonda, Birmingham, AL (US)

(21) Appl. No.: 18/685,838

(22) PCT Filed: Aug. 23, 2022
(86) PCT No.: PCT/US2022/075348

§ 371 Date: Feb. 22, 2024

Related U.S. Application Data
(60) Provisional application No. 63/236,187, filed on Aug. 23, 2021.
(60) Provisional application No. 63/341,253, filed on May 12, 2022.

(51) Int. Cl. A61K 51/08; A61K 9/00; A61K 47/64; A61P 17/00; A61P 29/00; C07K 7/08; C07K 7/64; C07K 14/00

(52) U.S. Cl. CPC A61K 51/088; A61K 9/0014; A61K 47/64; A61P 17/00; A61P 29/00; C07K 7/08; C07K 7/64; C07K 14/001; A61K 2121/00

(57) ABSTRACT

Disclosed herein are leukocyte-targeting molecules, pharmaceutical compositions comprising leukocyte-targeting molecules and an optional pharmaceutical agent, and methods of using same. In some embodiments, a leukocyte-targeting molecule is a peptide having the formula: X1X2AAX3AX4X5X17AX6X7X8AX9X10A(P)nX11x12(X13)n, or a pharmaceutically acceptable salt thereof: wherein X1, X2, X11, and X12 are each, independently, lysine, arginine, or ornithine; X3, X4, X5, X6, X7, X8, X9, and X10 are each, independently, valine, leucine, isoleucine, or norleucine; X13 is tyrosine; X17 is praline or alanine; and n is 0 or 1.

### CROSS REFERENCE TO RELATED APPLICATIONS

This application claims the benefit of U.S. Provisional Patent Applications 63/236,187 filed Aug. 23, 2021 and 63/341,253 filed May 12, 2022; the entire contents of which are incorporated by reference herein.

### FIELD

The claimed invention is directed to leukocyte-targeting molecules and pharmaceutical compositions comprising the same.

### SUMMARY

Disclosed herein are leukocyte-targeting molecules and compositions comprising a leukocyte-targeting molecule and an optional pharmaceutical agent.

In some embodiments disclosed herein, a leukocyte-targeting molecule is provided comprising a peptide having the formula:

(SEQ ID NO: 10)
X¹X²AAX³AX⁴X⁵X¹⁷AX⁶X⁷X⁸AX⁹X¹⁰A(P)ₙ¹¹X¹²(X¹³)ₙ

or a pharmaceutically acceptable salt thereof; wherein X¹, X², X¹¹, and X¹² are each, independently, lysine, arginine, or ornithine; X³, X⁴, X⁵, X⁶, X⁷, X⁸, X⁹, and X¹⁰ are each, independently, valine, leucine, isoleucine, or norleucine; X¹³ is tyrosine; X¹⁷ is proline or alanine; and n is 0 or 1. In some embodiments, X³ and X⁶ are valine. In some embodiments, X⁴, X⁵, X⁷, X⁸, X⁹, and X¹⁰ are each, independently, leucine, isoleucine, or norleucine.

In some embodiments, the leukocyte-targeting molecule has a formula:

(SEQ ID NO: 11)
X¹X²AAVALLPAVLLALLA(P)ₙX¹¹X¹²(X¹³)n

or a pharmaceutically acceptable salt thereof.

In some embodiments, the leukocyte-targeting molecule comprises at least one ornithine, isoleucine, or norleucine.

In some embodiments, the leukocyte-targeting molecule comprises an additional 1-5 amino acids on one or both of the C-terminal and N-terminal ends of the peptide. In some embodiments, the additional amino acid is a basic amino acid.

In some embodiments, the leukocyte-targeting molecule has an amino acid sequence selected from KKAAVALLPAVLLALLAKK (SEQ ID NO:5), RRAAVALLPAVLLALLARR (SEQ ID NO:6), RRAAVALLPAVLLALLARK (SEQ ID NO:7), RKAAVALLPAVLLALLARKY (SEQ ID NO:8), AAVALLPAVLLALLAPCVQRKRQKLMPC (SEQ ID NO:9), AAVALLPAVLLALLAPVQRKRQKLMP (SEQ ID NO:13), KKAAVALLPAVLLALLAPKK (SEQ ID NO:39), RRAAVALLPAVLLALLAPRR (SEQ ID NO:40), RRAAVALLPAVLLALLAPRK (SEQ ID NO:41), or RKAAVALLPAVLLALLAPRKY (SEQ ID NO: 42).

In some embodiments, the peptide further comprises the amino acid sequence CVQRKRQKLMPC (SEQ ID NO:38) at the carboxy terminus. In some embodiments, the peptide has a formula:

(SEQ ID NO: 12)
X¹X²AAX³AX⁴X⁵X¹⁷AX⁶X⁷X⁸AX⁹X¹⁰A(P)ₙX¹¹X¹²(X¹³)ₙCVQRKRQK
LMPC

or a pharmaceutically acceptable salt thereof.

In some embodiments, the compound comprises at least one radioisotope of iodine. In some embodiments, the peptide is iodinated. In some embodiments, the at least one radioisotope of iodine is selected, independently, from ¹²³I, ¹²⁴I, ¹²⁵I, or ¹³¹I, or the peptide is iodinated with ¹²³I, ¹²⁴I, ¹²⁵I, or ¹³¹I.

In some embodiments, the peptide comprises at least one D-amino acid.

Also disclosed herein is a composition comprising a compound disclosed herein. In some embodiments, the composition is a pharmaceutical composition further comprising a pharmaceutically acceptable excipient.

Also disclosed herein are methods of delivering an active pharmaceutical agent to a leukocyte, a CD34⁺ stem cell, or both, in a subject in need thereof, comprising administering a leukocyte-targeting molecule or composition disclosed herein. In some embodiments, the leukocyte is an eosinophil, basophil, neutrophil, or monocyte.

Also disclosed herein are methods of treating a disease in a subject, comprising administering a therapeutically effective amount of a leukocyte-targeting molecule or composition disclosed herein. In some embodiments, the disease is an inflammatory disease. In some embodiments, the disease is an autoimmune disease.

In some embodiments, the disease is a skin disorder. In some embodiments, the skin disorder is atopic dermatitis, psoriasis, rosacea, or acne. In some embodiments, the skin disorder is atopic dermatitis.

In some embodiments, the disease is chronic cutaneous lupus or systemic lupus erythematosus. In some embodiments, the disease is a viral disease. In some embodiments, the disease is shingles, herpes simplex type 1 or 2, or severe acute respiratory virus 2 (SARS-CoV-2).

In some embodiments, the autoimmune disease is rheumatoid arthritis, an inflammatory bowel disease, asthma, or type 1 diabetes. In some embodiments, the inflammatory bowel disease is Crohn's disease or ulcerative colitis.

In some embodiments, the disease is atherosclerosis, non-alcoholic steatohepatitis, or hypercholesteremia. In some embodiments, the disease is age-related macular degeneration, diabetic retinopathy, conjunctivitis, uveitis, or chronic sinusitis.

In some embodiments, the leukocyte-targeting molecule or composition is administered topically, orally, or by injection. In some embodiments, topical administration comprises topical creams, controlled release topical patches, eye drops, and nasal sprays.

Also disclosed herein are methods of reducing aberrant cytokine signaling in a subject in need thereof, comprising administering a leukocyte-targeting molecule or composition disclosed herein. In some embodiments, wherein the active pharmaceutical ingredient is delivered preferentially to leukocytes, CD34⁺ stem cells, or both, over red blood cells. In some embodiments, the leukocytes are eosinophils, basophils, neutrophils, or monocytes.

### BRIEF DESCRIPTION OF THE DRAWINGS

FIG. 1A-B depicts a comparison of concentrations of radioactivity (expressed as μg equivalents/mL) following single subcutaneous (FIG. 1A) and intravenous (FIG. 1B) administrations of [³H]-AMTX-100 to male albino rats at a target dose of 5 mg/kg. Plasma refers to whole blood depleted of red blood cells.

FIG. 2 depicts plasma levels of (3H)-AMTX-100 in plasma after administration by intravenous, subcutaneous, intratracheal, introduodenal, esophageal, or topical (skin) routes. All studies use L-amino acid version of (3H)-AMTX-100 except for the intraduodenal administration which used D-amino acids.

FIG. 3A-B depicts AMTX-100-FITC median fluorescence intensity in mouse leukocytes on two scales. FIG. 3A depicts intensity on a scale of 0-6,000 to provide intensity of eosinophils and FIG. 3B depicts intensity on a scale of 0-3,000 to provide intensity of other white blood cell populations.

FIG. 4A-B depict AMTX-100-FITC median fluorescence intensity in human leukocytes on two scales. FIG. 4A depicts intensity on a scale of 0-300,000 to provide intensity of eosinophils and FIG. 4B depicts intensity on a scale of 0-10,000 to provide intensity of other white blood cell populations.

FIG. 5A-B depicts distribution of radioactivity in rat tissues at 2 hr (FIG. 5A) and 12 hr (FIG. 5B) following a single intravenous administration of [³H]-AMTX-100 at a dose of 5 mg/kg.

FIG. 6 depicts a comparison of concentrations of radioactivity (expressed as ng equivalents/mL) following single topical administration of [³H]-AMTX-100 to male albino rats at a target dose of 5 mg/kg.

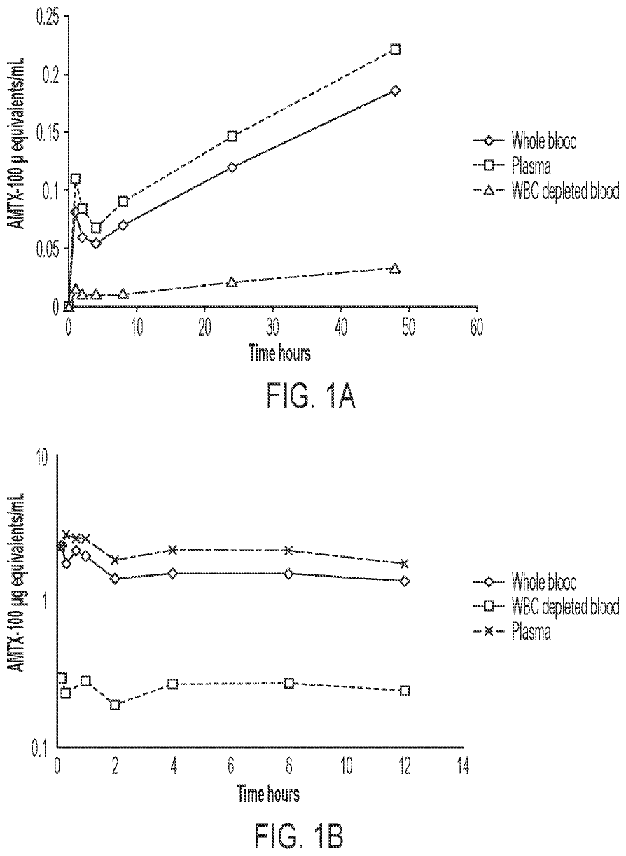

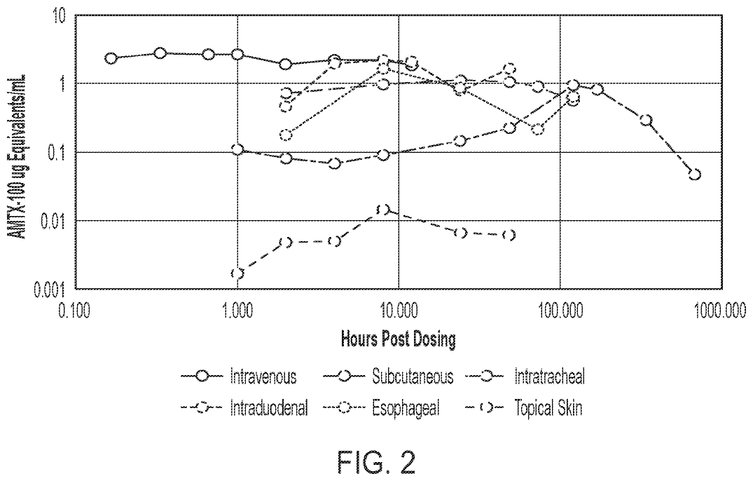

### DETAILED DESCRIPTION

AMTX-100 is a small peptide as disclosed in WO2001/37821 incorporated by reference herein in its entirety. It is a chimera, not found in nature, with a cell-penetrating amino terminus (sometimes called the signal sequence hydrophobic region or SSHR). The SSHR is linked to a stress response transcription factor (SRTFs) at the carboxy terminal end to yield AMTX-100. SRTFs contain a nuclear localization sequence (NLS) from NF-kB that permits it to bind to an Importin α/Importin β nuclear transporter complex. The amino terminus also contains a NLS found in carbohydrate and lipid metabolism promoting transcription factors. While molecules with the SSHR can penetrate cells, deleting the SSHR nullifies cell penetration. In other words, the NF-kB sequences alone cannot penetrate cells. AMTX-100 has three significant properties: 1) cell penetration, 2) modulation of the SRTF involved in the inflammatory pathway by a nuclear transporter, and 3) modulation of large transcription factors involved in metabolic pathways by a nuclear transporter. This provides for a competition between the fully functional transcription factors with a NLS with a similar NLS contained in AMTX-100. This competition modulates how much transcription factor gets transported into the nucleus to initiate transcription.

The transporter for AMTX-100 is Importin-α that complexes with Importin-β (Imp α5 and Imp β1, respectively). Imp α5 has a docking site for the NLS of a number of SRTFs and is the adaptor protein that recognizes a docking site on Imp β1, the functional element that enables crossing the nuclear membrane. The interaction between Imp α5 and Imp β1 permits the SRTF bound to Imp α5 to be transported through the nuclear pore.

Impβ1, in addition to binding and transporting SRTFs bound to Imp α5, has a second function. Imp 31 also recognizes an NLS in TFs needed for lipid (SREBP) and carbohydrate (ChREBP) metabolism that is distinct from the NLS of SRTFs. Imp β1 can transport these TFs to the nucleus without an adaptor protein.

Cells which are activated to be proinflammatory and produce SRTFs also need energy and lipids to proliferate and carry out their functions (cytokine, chemokine and growth factor production). Thus, AMTX-100 sequences can competitively modulate the NLS binding domains of SRTFs, ChREBP, and SREBP in a natural way, acting as a decoy for the TF docking sites at two important checkpoints in inflammation and metabolism.

The amino terminus of AMTX-100 is derived from the fibroblast growth factor (FGF)-4 leader sequence which was believed to permit FGF-4 to enter the endoplasmic reticulum for processing and extrusion of FGF-4 from the cell. FGF-4 outside the cell binds promiscuously to a variety of FGF receptors (1-4) and is aided by heparin. Binding of FGF-4 to its cell surface receptor invokes a tyrosine kinase cascade which brings about signaling within the cell.

It was originally believed that the SSHR on AMTX-100 could enter cells without a receptor, energy or endocytosis. However, in studies performed by the present inventors, an AMTX-100-related peptide with a double-null knockout for Importin α/Importin β complex and Importin β obtained by amino acid substitution and also containing a FITC molecule (FITC-SMTX-100), was incubated with human blood and analyzed by flow cytometry. This FITC-coupled, AMTX-100-related peptide appears to only enter leukocytes (white blood cells [WBCs]) and not red blood cells (RBCs); the first indication of cell specificity. In pharmacokinetic (PK) studies performed in mice, it was not possible to detect AMTX-100 in plasma depleted of RBCs and WBCs using LC-MS, sensitive to 2 ng/ml. Therefore, this suggested that AMTX-100 may traffic in the WBCs and be transmitted to other cells or be able to influence other cells indirectly through byproducts of its interaction with leukocytes.

Intraperitoneal injections of AMTX-100 have shown efficacy in a number of animal models of lung, liver, sepsis, and metabolic syndrome induced inflammation (see WO2001/037821, WO2013/052813, WO2018/232383, and WO2020/056250 which are incorporated by reference herein for all they disclose regarding AMTX-100 and inflammation). The peritoneal area is known to harbor leukocytes. Most leukocytes are mobile in blood and lymph and some are fixed in specific tissues.

Pharmacokinetics/biodistribution studies have been conducted with a radioactive form of AMTX-100 ([³H]-AMTX-100) in rats. The T_{1/2} for AMTX-100 in blood was 45-51 hours and the radiation was partitioned to the WBCs. [³H]-AMTX-100 was localized to organs where immune cells are known to reside and traffic through (spleen, lung, liver, lymph nodes, kidney, adrenal gland, bone marrow, pancreas, small intestine, etc.). Surprisingly absent was the lack of radioactivity localization in heart and other muscle cells. This suggests that AMTX-100 is not going into every organ and cell, which is contrary to published information.

Because there appeared to be a concentration of radioactivity in organs with immune cells, a series of flow cytometry experiments was initiated with human blood. All cells were first stained with FITC-AMTX-100 and then incubated with immunotyping reagents that separated hematopoietic cells into broad categories of RBCs, granulocytes, monocytes, B- and T-lymphocytes, and NK cells. There was no double-staining of RBCs confirming what was observed with human blood which was incubated with FITC-AMTX-100. In contrast, granulocytes and a subset of monocytes were identified to be double stained indicating that there was specificity within the leukocyte markers tested. Also double stained were a small subset of CD34+ hematopoietic stem cells. Again, a highly unexpected finding for a peptide which is “purported” to be a promiscuous, cell-penetrating, leader sequence.

Fibroblast growth factors are very important molecules in the development of limb formation and cell proliferation of organisms and are well studied. There are no reports in the literature of FGF leader sequences demonstrating cell penetration and specificity for cells, in particular those of FGF-4. Not all FGFs have a leader sequence. The inventors recognized that this finding of specificity for the FGF-4 leader sequence has important medical and industrial use as a highly specific linked or encapsulated drug delivery vehicle for targeting important immune cells.

Also disclosed herein are non-natural forms of the leader sequence of FGF-4 to couple with peptides, proteins, small molecule drugs, RNA, DNA, and radioactive isotopes. There are additional FGFs with leader sequences which may have unique biological activity and cell specificity, as found for the FGF4 leader sequence.

### Compositions

Disclosed herein are compositions comprising a molecule capable of targeting white blood cells (WBC, leukocytes) and CD34⁺ stem cells and a therapeutic, diagnostic, or prophylactic pharmaceutical agent. Leukocytes are a type of blood cell that is made in the bone marrow and found in the blood and lymph tissue and are part of the body's immune system. They help the body fight infection and other diseases. Types of white blood cells (leukocytes) are granulocytes (neutrophils, eosinophils, and basophils), monocytes, and lymphocytes (T cells, B cells, and NK cells).

In some embodiments, the compositions include a compound provided herein, such as a leukocyte-targeting molecule, and a pharmaceutical agent, such as a therapeutic, diagnostic, or prophylactic agent, wherein the compound and pharmaceutical agent may be covalently or non-covalently linked. The compositions disclosed herein can be tailored to delivery pharmaceutical agents to the cytoplasm or nucleus of the cell. The compositions disclosed herein can also comprise the leukocyte-targeting molecule fused to a protein or peptide pharmaceutical agent.

The leukocyte-targeting molecules disclosed herein comprises a cell-penetrating amino terminus from a fibroblast growth factor sequence (an FGF signal sequence, alternatively referred to as a signal sequence hydrophobic region or SSHR). In some embodiments, the FGF signal sequence is linked to a stress response transcription factor (SRTF) nuclear localization sequence (NLS) at the carboxy terminal end. In some embodiments, the SRTF contains a NLS from NF-kB that permits it to bind to an Importin α/Importin β nuclear transporter complex. The amino terminus also contains a NLS found in carbohydrate and lipid metabolism promoting transcription factors. In some embodiments, the leukocyte-targeting molecule includes the cell-penetrating amino terminus linked to peptide, nucleic acid, or radioisotope pharmaceutical agent.

In some embodiments, the leukocyte-targeting molecule is derived from a fibroblast growth factor (FGF) signal sequence, for example based on the underlined FGF sequence of SEQ ID NO:1. Variations of the FGF4 signal sequence are also useful in the claimed compositions as depicted in Table 1. In some embodiments, the leukocyte-targeting molecule sequence includes a modification to knock-out Importin β activity. In some embodiments, the leukocyte-targeting molecule sequence maintains Importin β activity

FGF4 (Signal sequence is underlined):

(SEQ ID NO: 1)
MSGPGTAAVA LLPAVLLALL APWAGRGGAA APTAPNGTLE
AELERRWESL VALSLARLPV AAQPKEAAVQ SGAGDYLLGI
KRLRRLYCNV GIGFHLQALP DGRIGGAHAD TRDSLLELSP
VERGVVSIFG VASRFFVAMS SKGKLYGSPF FTDECTFKEI
LLPNNYNAYE SYKYPGMFIA LSKNGKTKKG NRVSPTMKVT
HFLPRL

TABLE 1
Leukocyte-targeting molecules

| SEQ ID | | |
|---|---|---|
| NO: | SEQUENCE | Comments |
| 2 | AAVAPAVX¹⁶X¹⁶AX¹⁶X¹⁶A | |
| 3 | AAVALLPAVLLALLAP | FGF4 leader |
| | | sequence |
| 4 | X¹X¹AAVALLPAVLLALLAX¹X¹ | |
| 5 | KKAAVALLPAVLLALLAKK | |
| 6 | RRAAVALLPAVLLALLARR | |
| 7 | RRAAVALLPAVLLALLARK | |
| 8 | RKAAVALLPAVLLALLARKY | |
| 9 | AAVALLPAVLLALLAPCVQRKRQKLMPC | AMTX-100; |
| | | cSN50.1 |
| 10 | X¹X²AAX³AX⁴X⁵X¹⁷AX⁶X⁷X⁸AX⁹X¹⁰A(P)ₙX¹¹X¹²(X¹³)ₙ | |
| 11 | X¹X²AAVALLPAVLLALLAPX¹¹X¹²(X¹³)ₙ | |
| 12 | X¹X²AAX³AX⁴X⁵X¹⁷AX⁶X⁷X⁸AX⁹X¹⁰APX¹¹X¹²(X¹³)ₙCVQRKRQKLMPC | |
| 13 | AAVALLPAVLLALLAPVQRKRQKLMP | SN50 |
| 14 | AAVALLPAVLLALLAPCYVQRKRQKLMP | cSN50 |
| 15 | AAVAPAVX¹⁶X¹⁶AX¹⁶X¹⁶AP | |
| 16 | AAVALLPAVLLALLAP | Human core |
| | | SSHR |
| 17 | (X¹⁴)ₙ(X¹⁵)ₙAAVALLP(X¹⁴)ₘ | |
| 18 | (X¹⁴)ₙ(X¹⁵)ₙAVLLALLAP(X¹⁴)ₘ | |
| 20 | (X¹⁴)ₙ(X¹⁵)ₙAAVALLAAVLLALLAP(X¹⁴)ₘ | |
| 21 | TTGTLLPGVLLALVVA | Murine core |
| | | SSHR |
| 22 | (X¹⁴)ₙ(X¹⁵)ₙTTGTLLPGVLLALVVA(X¹⁴)ₘ | |
| 23 | TTGTLLPRVLLALVVA | Rat core |
| | | SSHR |
| 24 | (X¹⁴)ₙ(X¹⁵)ₙTTGTLLPGVLLALVVA(X¹⁴)ₘ | |
| 25 | (X¹⁴)ₙ(X¹⁵)ₙTTGTLLP(X¹⁴)ₘ | |
| 26 | (X¹⁴)ₙ(X¹⁵)ₙVLLALVVA(X¹⁴)ₘ | |
| 28 | RAAVALLPAVLLALLAPY(X¹⁴)ₘ | |
| 29 | RAAVALLPAVLLAVLAPY(X¹⁴)ₘ | |
| 30 | (X¹⁴)ₙ(X¹⁵)ₙAAVALLPAVLLALLAPDVRKRQDLEQKM(X¹⁴)ₙ(Y)ₘ | |
| 31 | (X¹⁴)ₙ(X¹⁵)ₙAAVALLPAVLLALLAP(X¹⁴)ₘ | |
| 32 | (X¹⁴)ₙ(X¹⁵)ₙAAVALLAAVLLALLAP(X¹⁴)ₘ | |
| 33 | (X¹⁴)ₙ(X¹⁵)ₙTTGTLLPGVLLALVVA(X¹⁴)ₘ | |
| 34 | (X¹⁴)ₙ(X¹⁵)ₙTTGTLLPRVLLALVVA(X¹⁴)ₘ | |
| 35 | (X¹⁴)ₙ(X¹⁵)ₙTTGTLLP(X¹⁴)ₘ | |
| 36 | (X¹⁴)ₙ(X¹⁵)ₙGVLLALVVA(X¹⁴)ₘ | |
| 37 | (X¹⁴)ₙ(X¹⁵)ₙAAVALLPAVLLALLAPCVQKRQKLMPC | |
| 39 | KKAAVALLPAVLLALLAPKK | |
| 40 | RRAAVALLPAVLLALLAPRR | |
| 41 | RRAAVALLPAVLLALLAPRK | |
| 42 | RKAAVALLPAVLLALLAPRKY | |
| 43 | KRAAVALLPKK | |
| 44 | KRAVLLALLAPKK | |
| 45 | KRAAVALLAAVLLALLAPKK | |
| 46 | KRTTGTLLPGVLLALVVAKK | |
| 47 | KRTTGTLLPGVLLALVVAKK | |
| 48 | KRTTGTLLPKK | |
| 49 | KRVLLALVVAKK | |
| 50 | KRAAVALLPKK | |
| 51 | AAVALLPAVLLAVLAPCVQRKRQKLMPC | cSN50.1α |
| 52 | AAVALLPAVLLALLAPCVQRDEQKLMPC | cSN50.1β |
| 19 | AAVALLPAVLLAVLAPCVQRDEQKLMPC | Null cSN50.1 |

X¹, X², X¹¹, and X¹² = Lys, Arg, or Orn
X³, X⁴, X⁵, X⁶, X⁷, X⁸, X⁹, X¹⁰ = Val, Leu, Ile, or Nle
X¹³ = Tyr;
X¹⁴ = Lys;
X¹⁵ = Arg;
X¹⁶ = Leu, Ile, or Nle;
X¹⁷ = Pro or Ala.
ₙ = 0 or 1
ₘ = 0 or 2

In some embodiments, the peptides of Table 1 are linear. In some embodiments, SEQ ID NOs. 9, 10, 12, 14, and 37 are optionally cyclized at the cysteine residues. In some embodiments, the sequences are cyclized.

In some embodiments, the leukocyte-targeting molecule is

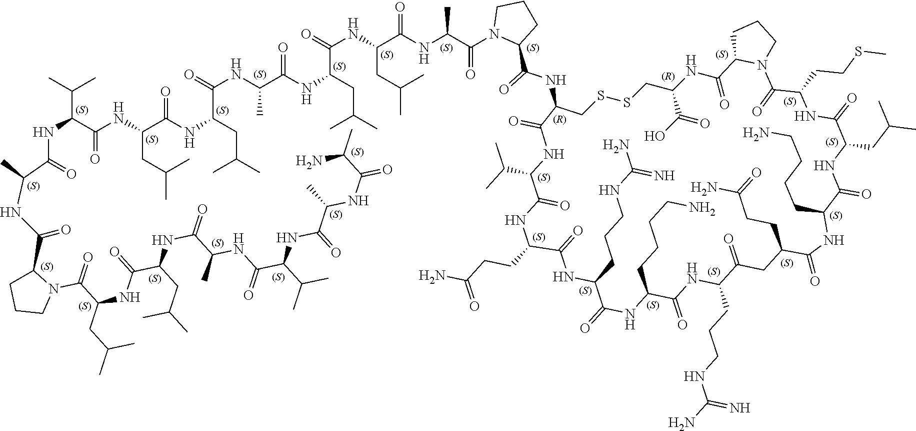

or a salt thereof.

In some embodiments, the pharmaceutical agent is a peptide, a protein, a nucleic acid, a small molecule, a heavy metal, an imaging agent, or a radioactive agent. In some embodiments, the pharmaceutical agent is an enzyme comprising, but not limited to, horseradish peroxidase or alkaline phosphatase. In some embodiments, the heavy metal comprises, but is not limited to, colloidal gold or gadolinium. In some embodiments, the radioactive agent comprises, but is not limited to, ³H-, ¹⁴C-, ¹²⁵I-, ¹⁸F- or ¹³¹I-. In some embodiments, the imaging agent is a radioactive agent or a fluorescent label. In some embodiments, the fluorescent label comprises, but is not limited to, fluorescein, rhodamine, or green fluorescent protein, or derivatives thereof.

In some embodiments, the leukocyte-targeting molecule and the pharmaceutical agent are encapsulated in a micelle or a liposome. In some embodiments, the leukocyte-targeting molecule is incorporated into a micelle or liposome such that the leukocyte-targeting molecule is available on the surface of the micelle or liposome for interaction with leukocytes.

In some embodiments, the leukocyte-targeting molecules specifically target eosinophils, basophils, neutrophils, and monocytes. In some embodiments, the leukocyte-targeting molecules do not target T-lymphocytes, B-lymphocytes, or NK cells.

The compositions disclosed herein can optionally include coupling the leukocyte-targeting molecules with a antibody to allow bispecific targeting. These antibody-coupled molecules would target leukocytes by the disclosed leukocyte-targeting molecule and a second specificity would be targeting via the antibody. These bispecific molecules, depending on the antibody specificity, interfere with signaling pathways involved in tumor development, and redirect or recruit immune cells to the target tissue.

### Methods of Use

The compositions disclosed herein are useful for targeting of diagnostic, therapeutic, and prophylactic agents to leukocytes. In some embodiments, the leukocytes are targeted in a subject in need of treatment for an autoimmune, inflammatory, or neurodegenerative disease, or in need of reduction of aberrant cytokine production.

As disclosed herein, an inflammatory disease is a disease or disorder including, but not limited to, acute disseminated encephalomyelitis (ADEM), Addison's disease, an allergy, allergic rhinitis, Alzheimer's disease, anti-phospholipid antibody syndrome (APS), an arthritis such as, e.g., a monoarthritis, an oligoarthritis, or a polyarthritis like an osteoarthritis, a rheumatoid arthritis, a juvenile idiopathic arthritis, a septic arthritis, spondyloarthropathy, gout, pseudogout, Still's disease, asthma, autoimmune hemolytic anemia, autoimmune hepatitis, an autoimmune inner ear disease, bullous pemphigoid, celiac disease, Chagas disease, chronic obstructive pulmonary disease (COPD), diabetes mellitus type 1 (IDDM), endometriosis, a gastrointestinal disorder such as, e.g., an irritable bowel disease or an inflammatory bowel disease such as Crohn's disease or ulcerative colitis, Goodpasture's syndrome, Graves' disease, Guillain-Barre syndrome (GBS), Hashimoto's thyroiditis, hidradenitis suppurativa, idiopathic thrombocytopenia purpura, interstitial cystitis, a lupus, such as, e.g., a discoid lupus erythematosus, a drug-induced lupus erythematosus, a lupus nephritis, a neonatal lupus, a subacute cutaneous lupus erythematosus, or a systemic lupus erythematosus, morphea, multiple sclerosis (MS), myasthenia gravis, a myopathy such as, e.g., a dermatomyositis, an inclusion body myositis, or a polymyositis, myositis, narcolepsy, neuromyotonia, Parkinson's disease, pemphigus vulgaris, pernicious anaemia, primary biliary cirrhosis, psoriasis, recurrent disseminated encephalomyelitis, rheumatic fever, scleroderma, Sjögren's syndrome, a skin disorder such as, e.g., dermatitis, eczema, statis dermatitis, atopic dermatitis, hidradenitis suppurativa, psoriasis, rosacea, or scleroderma, tenosynovitis, uveitis, vasculitis such as, e.g., Buerger's disease, cerebral vasculitis, Churg-Strauss arteritis, cryoglobulinemia, essential cryoglobulinemic vasculitis, giant cell arteritis, Golfer's vasculitis, Henoch-Schonlein purpura, hypersensitivity vasculitis, Kawasaki disease, microscopic polyarteritis/polyangiitis, polyarteritis nodosa, polymyalgia rheumatica (PMR), rheumatoid vasculitis, Takayasu arteritis, Wegener's granulomatosis, alopecia areata, or vitiligo.

In some embodiments, the autoimmune or inflammatory disease is a skin disorder. In some embodiments, the skin disorder is psoriasis. In some embodiments, the skin disorder is atopic dermatitis. In some embodiments, the skin disorder is treated with a topical administration of a compound, composition, or leukocyte-targeting molecule disclosed herein.

As disclosed herein, a neurodegenerative disease is a disease or disorder including, but not limited to, Parkinson's disease, Alzheimer's disease, multiple sclerosis, optic neuritis, stroke, CNS trauma, amyotrophic lateral sclerosis, a neuropathy, a nervous system hypoxia, a CNS toxicity, dementia, retinopathy, Huntington's disease, synucleinopathy, epilepsy, autism, and an aging-related CNS degeneration. In some embodiments, the neurodegenerative disease is Alzheimer's disease.

In some embodiments, the aberrant cytokine production is cytokine release syndrome (CRS). CRS is the aberrant overproduction of proinflammatory cytokines such as INF-γ, IL-1α, IL-1β, IL-6, IL-8, GM-CSF, M-CSF, and TNF-α resulting in high concentrations of systemic circulating cytokines. In some embodiments disclosed herein, a composition disclosed herein can reduce production of proinflammatory cytokines.

The administration of a composition disclosed herein may be by any suitable means that results in uptake of the composition into a white blood cell or CD34⁺ stem cell. The composition may be contained in any appropriate amount in any suitable carrier substance, and is generally present in an amount of 1-95% by weight of the total weight of the composition. The composition may be provided in a dosage form that is suitable for local or systemic administration (e.g., parenteral, subcutaneously, intravenously, intramuscularly, or intraperitoneally). The pharmaceutical compositions may be formulated according to conventional pharmaceutical practice (see, e.g., (Gennaro, A. R. ed. (2000) Remington: The Science and Practice of Pharmacy (20th ed.), Lippincott Williams & Wilkins, Baltimore, Md.; Swarbrick, J. and Boylan, J. C. eds. (1988-1999) Encyclopedia of Pharmaceutical Technology, Marcel Dekker, New York).

Compositions as described herein may be administered parenterally by injection, infusion, topical application, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. In one embodiment, a composition as described herein is administered via osmotic pump. The composition may be administered orally in sublingual form or with a coating protecting the composition from gastrointestinal peptidases. The formulation and preparation of such compositions are well known to those skilled in the art of pharmaceutical formulation. Formulations can be found in Gennaro supra.

Compositions for parenteral use may be provided in unit dosage forms (e.g., in single-dose ampoules), or in vials containing several doses and in which a suitable preservative may be added. The composition may be in the form of a solution, a suspension, an emulsion, an infusion device, or a delivery device for implantation, or it may be presented as a dry powder to be reconstituted with water or another suitable vehicle before use. Apart from the active agent that treats or prevents inflammation, for example, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions described herein may be in a form suitable for sterile injection. To prepare such a composition, the suitable active therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine), and poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active ingredient (e.g., drug) in a predetermined pattern (e.g., in order to achieve a controlled release formulation) or it may be adapted not to release the active ingredient until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Swarbrick J. and Boylan, J. C. supra. Two or more compounds may be mixed together in the tablet, or may be partitioned. In one example, the first active therapeutic is contained on the inside of the tablet, and the second active therapeutic is on the outside, such that a substantial portion of the second active therapeutic is released prior to the release of the first active therapeutic. Therapeutic combinations that decrease the level of inflammation, for example, are identified as useful in the compositions, methods, and kits described herein.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Compositions as described herein can also be formulated for inhalation, topical application, and intravitreal injection. Combinations are expected to be advantageously synergistic. Topical formulations include creams or ointments as well as controlled release patches which delivery a composition disclosed herein through the skin.

The therapeutic methods described herein in general include administration of a therapeutically effective amount of a composition described herein to a subject (e.g., animal) in need thereof, including a mammal. In some embodiments, the subject is a human. In some embodiments, the subject is a non-human mammal. Such treatment will be suitably administered to subjects in need thereof. Determination of those subjects “in need thereof” can be made by any objective or subjective determination by a diagnostic test or opinion of a subject or health care provider.

### EXAMPLES

### Example 1. Pharmacokinetic/Biodistribution of AMTX-100

Formulation analysis. The radiochemical purity of [³H]-AMTX-100 in the formulation was assessed using high performance liquid chromatography (HPLC) with radiochemical detection prior to and after dose administration. This was conducted by injecting 50 μL of an aliquot of dose formulation on to the HPLC system.

Radioactivity concentration and homogeneity checks were conducted on each formulation prior to and after dose administration by direct quantitative radiochemical analysis.

Formulation administration. Each rat was weighed prior to dose administration and the individual doses administered were calculated based on body weight, target dose volume and the radioactive concentration of the dose formulation.

Administration was achieved with the aid of a butterfly needle, tubing and syringe. The formulation was administered directly into a tail vein. The dose volume (2.5 mL/kg) was used to achieve a dose level of 5 mg/kg and radioactive dose levels of 100 μCi/kg (Phase 1) and 500 μCi/kg (Phase 2). The dose formulation was stirred continuously throughout the dosing procedure.

Administration by volume. For Phases 1 and 2, an appropriate volume of dose formulation, including excess, was taken up into the dose utensil for each dose. Fresh dose utensils were used for each animal where possible. Any trapped air and the excess formulation was expelled leaving the dose utensils primed with the appropriate volume. The dose was administered and the radioactive dose received was calculated by the volume of dose dispensed and the calculated radioactive concentration of the dose formulation.

[³H]-NSP labelling. For the main phase of the study, the test protein was prepared, radiolabeled with N-succinimidyl-[2,3-3H]-propionate ([³H]-NSP) and purified as described below.

Three aliquots of 1.6 mL [³H]-NSP solution were dispensed into appropriate vials. The solvent was evaporated under a gentle stream of nitrogen gas at ambient temperature. Once enough of the solvent had been evaporated, the three aliquots were combined into one vial and the remaining solvent evaporated until dryness. The residue in the vial was re-suspended in 0.6 mL of 1 mg/mL AMTX-100 by gently vortex mixing to ensure complete mixing of the protein with the labelling reagent. The solutions were allowed to stand at ambient temperature for approximately 1 hour.

The [³H]-NSP-protein solutions were then purified by reverse phase chromatography techniques and the resulting solution was frozen and lyophilized using a freeze drier. The resulting material was dissolved in 2 mL ethanol:water (50:50) and stored at approximately −20° C. prior to formulation for dose preparation.

Following purification, the radiochemical purity and specific activity of the [³H]-labelled test protein were determined using reverse phase HPLC and quantitative radiochemical analysis (QRA).

During the method development for radio-labelling AMTX-100, additional analysis by LC-RAD-MS/MS of AMTX-100 and [³H]-AMTX-100, with and without incubation 1:1 with 15% hydrogen peroxide (5 minutes), was also performed to confirm labelling of the full length peptide.

The specific activity of the radiolabeled protein was calculated following liquid scintillation counting (LSC) of weighed aliquots of diluted sample. Specific activity was determined as μCi/mg of protein.

Formulation. The required quantities of each radiolabeled test item were combined with the corresponding non-labelled test item as appropriate to achieve a radioactive concentration sufficient to meet the aims of the study. Dose solutions were prepared in PBS buffer pH 7.4, with the intention of administering a total dose volume of 5 mg/kg, (2.5 ml/kg, 100 Ci/kg-Phase 1 and 2.5 ml/kg, 500 Ci/kg-Phase 2).

Quantitative Whole Body Autoradiography (QWBA). The frozen carcasses were subjected to QWBA. Sections were presented at up to five different levels of the rat body to include between 30 and 40 tissues (subject to presence of sufficient radioactivity).

The freeze-dried whole body autoradiography sections were exposed to phosphor-storage imaging plates and incubated at ambient temperature in the dark for seven days. Calibrated autoradiographic blood standards containing known amounts of radioactivity (nCi/g) were included in each exposure.

Distribution of radioactivity in tissues and [³H]-blood standards (prepared and validated at Pharmaron UK Ltd.) was determined and quantified using a Fuji FLA-5100 fluorescent image analyzing system and associated Tina (version 2.09) and SeeScan (version 2.0) software. For each exposure, a standard curve was produced in SeeScan using the data from the autoradiographic blood standards from which tissue concentrations of radioactivity (μg equivalents/g) were determined.

A representative background radioactivity measurement for each exposure plate used was also taken. The number of measurements for each particular tissue was determined by the number of levels it appeared in. Multiple measurements were therefore performed for larger tissues that appeared in more than one level (e.g. liver), with only one measurement obtained for small tissues (e.g. thyroid gland). The limit of accurate quantification was considered to be the lowest [³H]-blood standard visible.

Prior to analysis, samples were stored at approximately −20° C. (carcass) or ambient temperature (sections after freeze drying). After analysis carcass remains were retained at approximately −20° C. pending disposal, sections were stored at ambient temperature.

### Phase 1: Total Radioactivity in Plasma

Eight male Sprague Dawley rats each received a single IV administration of [³H]-AMTX-100 and a terminal sample of whole blood (each approximately 5-10 mL) was collected via cardiac puncture under isofluorane anesthesia from a single rat at the following time points: 10, 20 and 40 minutes and 1, 2, 4, 8 and 12 hours post-dose

Animals were killed by exsanguination of blood via cardiac puncture followed by immediate snap freezing the carcass in a hexane/dry ice mixture (approximately −70° C.) after the final blood collection. Each carcass was stored at approximately −20° C., pending analysis by QWBA.

Whole blood was collected into tubes containing K₂-EDTA as anticoagulant. Following collection, an aliquot of whole blood (approximately 4 mL) was retained for analysis of whole blood and white blood cell (WBC) concentrations. The remaining whole blood samples were processed to plasma. Plasma samples were not depleted of white blood cells.

### Phase 2: Tissue Distribution of Radioactivity

Three of the carcasses obtained from the eight male Sprague Dawley rats subjected to whole blood collection and analysis after each receiving a single intravenous administration of [³H]-AMTX-100, were selected for QWBA analysis at each of the following time points: 10 minutes and 2 and 12 hours post-dose.

Each carcass was snap frozen by immersion in a hexane/dry ice mixture immediately after collection and was then stored at approximately −20° C., pending analysis by QWBA.

TABLE 2
Concentrations of radioactivity in whole blood, plasma, white blood
cell depleted blood and recovered white blood cells (expressed as
μg equivalents/mL) following a single subcutaneous administration of
[³H]-AMTX-100 to male albino rats at a target dose of 5 mg/kg

| | AMTX-100 μg equivalents/mL¹ | | | | | |
|---|---|---|---|---|---|---|
| | 01M | 02M | 03M | 04M | 05M | 06M |
| Matrix | 1 h | 2 h | 4 h | 8 h | 24 h | 48 h |
| Whole Blood | 0.081 | 0.059³ | 0.054 | 0.070 | 0.120 | 0.186 |
| Plasma³ | 0.110 | 0.084 | 0.068 | 0.090 | 0.146 | 0.221 |
| WBC depleted blood | 0.015 | 0.011 | 0.011 | 0.011 | 0021 | 0.033 |
| Recovered White | BLQ | BLQ | BLQ | BLQ | BLQ | BLQ |
| Blood Cells² | | | | | | |

WBC White blood cell
h—hours
¹Assume 1 g blood/plasma is equivalent to 1 mL
²Reverse wash of filters - may not represent total white blood cells
³Plasma not depleted of white blood cells
BLQ Below limit of accurate quantification

TABLE 3
Blood partition calculation following a single intravenous administration
of [³H]-AMTX-100 to male albino rats at a target dose of 5 mg/kg

| Time | Blood | Plasma | Blood:plasma | % cell | Partition | Non-plasma |
|---|---|---|---|---|---|---|
| (h) | (μg/mL) | (μg/mL) | ratio | volume | coefficient | compartment |
| 1 | 0.081 | 0.110 | 0.736 | 0.35 | 88.3 | 11.7 |
| 2 | 0.059¹ | 0.084 | 0.702 | 0.44 | 79.7 | 20.3 |
| 4 | 0.054 | 0.068 | 0.794 | 0.40 | 75.6 | 24.4 |
| 8 | 0.070 | 0.090 | 0.778 | 0.40 | 77.1 | 22.9 |
| 24 | 0.120 | 0.146 | 0.822 | 0.40 | 73.0 | 27.0 |
| 48 | 0.186 | 0.221 | 0.842 | 0.40 | 71.3 | 28.7 |

TABLE 4
Pharmacokinetic parameters for total radioactivity in blood,
plasma and WBCD blood following a single subcutaneous administration
of [³H]-AMTX-100 to male non-pigmented rats at 5 mg/kg

| | | Whole | WBCD |
|---|---|---|---|
| Parameter | Plasma¹ | blood¹ | blood¹ |
| Tmax (h) | 48 | 48 | 48 |
| Cmax (μg/mL) | 0.221 | 0.186 | 0.033 |

Tmax—Time at which maximum concentration was apparent
Cmax—Maximum measured concentration
WBCD—White blood cell depleted

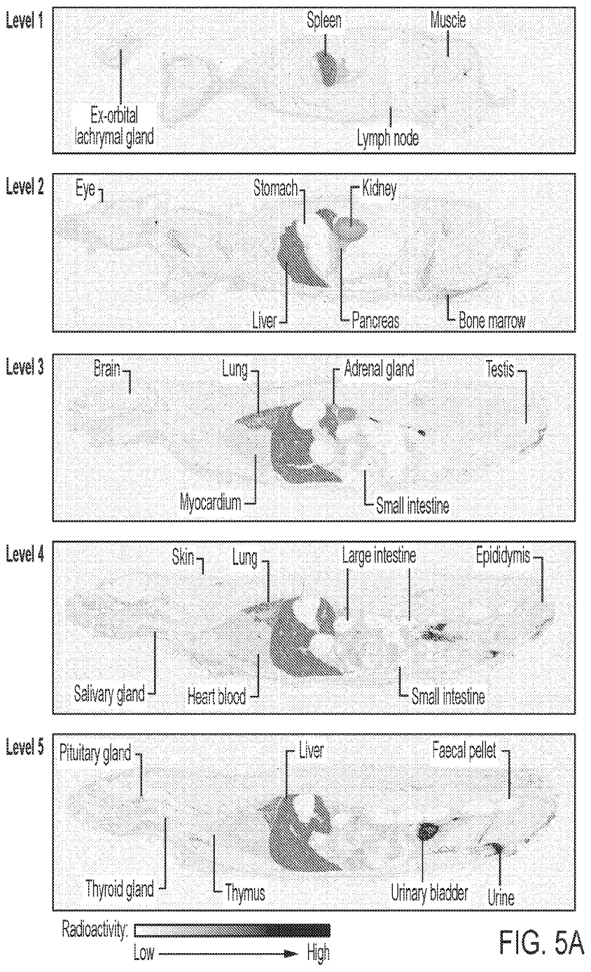

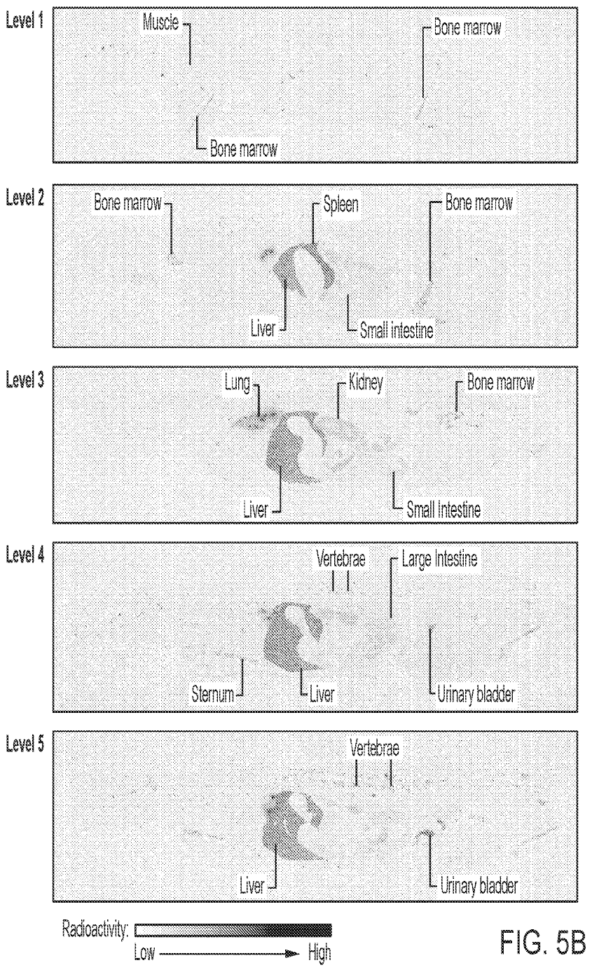

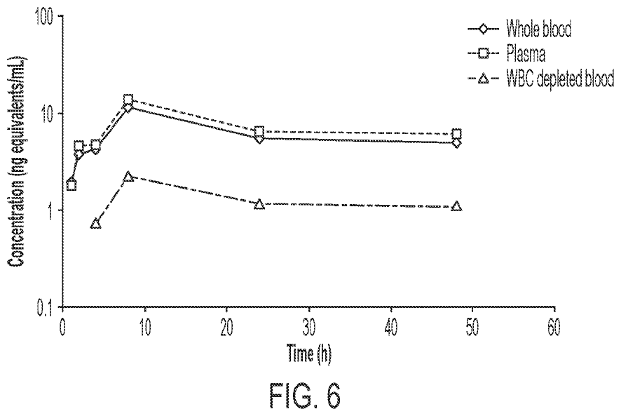

Distribution of radioactivity in rats following a single intravenous administration of [H]-AMTX-100 at a target dose of 5 mg/kg is depicted in FIG. 5A (2 hr) and FIG. 58 (12 hr) and in Table 5. Distribution of radioactivity in rats following a single topical administration of [H]-AMTX-100 at a target dose of 5 mg/kg is depicted in FIG. 6.

TABLE 5
Concentration of radioactivity in tissues (expressed as μg
equivalents/g) following a single intravenous administration of [
³H]-AMTX-100to male albino rats at a target dose of 5 mg/kg

| | | AMTX-100 μg equivalents | | |
|---|---|---|---|---|
| | | Animal no.: | | |
| | | 1M | 5M | 8M |
| | | Time point: | | |
| Tissue Type | Tissue | 10 min | 2 hr | 12 hr |
| Alimentary canal | Caecum contents | BLQ | BLQ | BLQ |
| | Caecum mucosa | 0.82 | 0.76 | ND |
| | Large intestine contents | BLQ | BLQ | 0.36 |
| | Large intestine mucosa | 0.80 | 0.63 | BLQ |
| | Small intestine contents | BLQ | 1.23 | 0.47 |
| | Small intestine mucosa | 0.54 | 0.89 | BLQ |
| | Stomach contents | BLQ | BLQ | ND |
| | Forestomach mucosa | 0.52 | 0.57 | ND |
| | Glandular stomach mucosa | 1.13 | 0.81 | ND |
| CNS | Brain | BLQ | BLQ | ND |
| | Choroid plexus | 0.71 | NS | NS |
| | Spinal cord | BLQ | BLQ | ND |
| Connective | Bone | BLQ | BLQ | ND |
| Dermal | Skin | 0.48 | 0.39 | ND |
| Endocrine | Adrenal gland | 9.93 | 5.33 | 0.44 |
| | Pituitary gland | 2.21 | 1.58 | ND |
| | Thyroid gland | 1.66 | 1.29 | ND |
| Excretory/metabolic | Liver | 58.60 | 31.60 | 8.77 |
| | Kidney: cortex | 16.30 | 9.59 | 0.61 |
| | Kidney: medulla | 10.10 | 4.47 | 0.45 |
| | Kidney: whole | 13.30 | 7.35 | 0.54 |
| | Urinary bladder contents | 67.50 | 219.00* | 11.90 |
| | Urinary bladder wall | 0.69 | 1.22 | BLQ |
| Exocrine | Ex-orbital lachrymal gland | 0.71 | 0.46 | ND |
| | Harderian gland | 0.46 | 0.37 | ND |
| | Pancreas | 1.71 | 1.69 | BLQ |
| | Salivary gland | 1.09 | 0.80 | ND |
| Fatty | Fat: brown | 0.59 | BLQ | ND |
| | Fat: white | 0.38 | BLQ | ND |
| Ocular | Eye: lens | BLQ | BLQ | ND |
| | Eye: whole | BLQ | BLQ | ND |
| Reproductive | Epididymis | 0.44 | 0.67 | ND |
| | Prostate gland | 0.41 | 0.83 | ND |
| | Testis | BLQ | 0.37 | BLQ |
| Respiratory | Lung | 96.70 | 31.90 | 30.10 |
| Skeletal/Muscular | Muscle | 0.52 | BLQ | BLQ |
| | Myocardium | 1.25 | 0.53 | ND |
| Vascular/Lymphatic | Blood: residual cardiac | 2.89 | 0.82 | BLQ |
| | Bone marror | 4.44 | 4.40 | 2.18 |
| | Lymph node | 1.13 | 0.83 | ND |
| | Spleen: marginal zone | 123.00 | 84.50 | 18.30 |
| | Spleen: red pulp | 15.40 | 11.90 | 6.89 |
| | Spleen: white pulp | 8.70 | 2.92 | 0.89 |
| | Spleen: whole | 55.80 | 41.80 | 11.90 |
| | Thymus | 0.45 | 0.68 | ND |

*Above upper limit of accurate quantification (>153 μg equivalents/g)
BLQ Below lower limit of accurate quantification (<0.36 μg equivalents/g)
ND Radioactivity not detected
NS No sample

### Example 2. Timeline of Plasma Appearance of AMTX-100 After Multiple Routes of Administration

AMTX-100 was radiolabeled with 3H at lysine residues and injected into Sprague-Dawley rats in a single dose by seven different routes (intravenous, subcutaneous, topical [skin], intratracheal, intraduodenal, esophageal, and oral). The rats were evaluated at various time points, up to 120 hours, by: a) blood samples were taken for pharmacokinetics (PK) by cardiac puncture; b) rats were frozen and sectioned for QWBA and/or micro-auto-radiography and histology; and c) frozen sections were made and freeze dried and exposed to phosphor-storage imaging plates and tissue radioactivity in images was quantified as tissue: blood ratios.

For all PK studies, the following definitions were used: WB=whole blood; Plasma=whole blood depleted of red blood cells; WBCD=whole blood depleted of white blood cells in which the whole blood is passed over a Pall Acrodisc filter (about 60% efficient); WBCs=the cells which bind to, and are washed off, the Pall Acrodisc filter.

[³H]-AMTX-100 by all routes of administration makes its way into the bloodstream and PK shows a very long residence time in plasma unexpected of a peptide by some routes (Sub-Q>672 hr or 28 days). WBCs and not RBCs are the specific target for [³H]-AMTX-100. Esophageal administration with L-amino acid version of [³H]-AMTX-100 is resistant to degradation was the D-amino acid form from the duodenal administration. The inventors hypothesize that the peptide very rapidly enters WBCs via a receptor, protecting it from extracellular enzymes. Persistence of radioactivity of intact [³H]-AMTX-100 in plasma remains relatively constant at 1 μg equivalents/ml after a single dose of [³H]-AMTX-100 even up to >120 hr and there is also a significant amount of [³H]-AMTX-100 found in tissue in which WBCs congregate, but not cardiac or skeletal muscle tissue. The biological effects are expected to be very long lasting in WBCs.

[³H]-AMTX-100 has a very long half-life (T_{1/2}) for a peptide (25-51 hr) after IV administration.

### Example 3. Flow Cytometry Studies

### Mouse

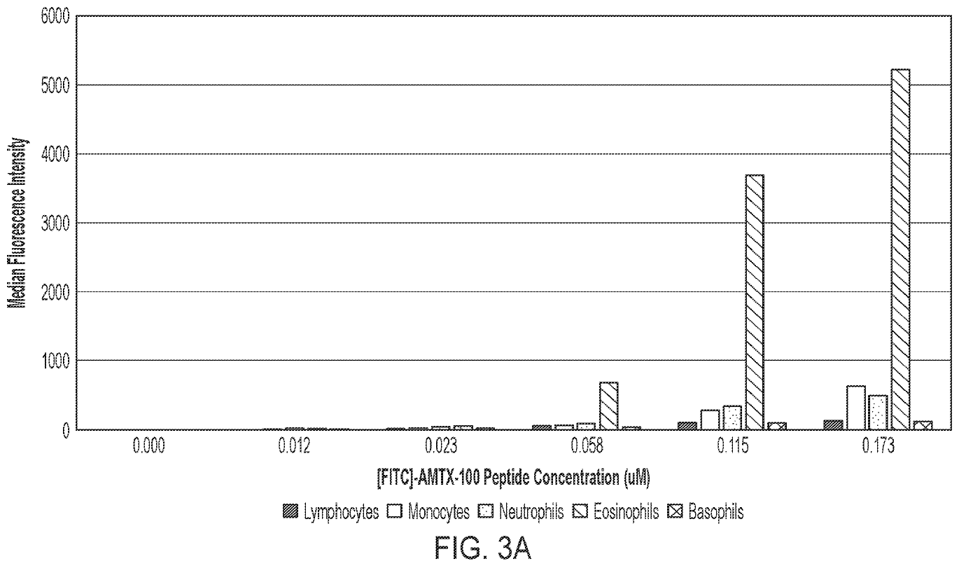

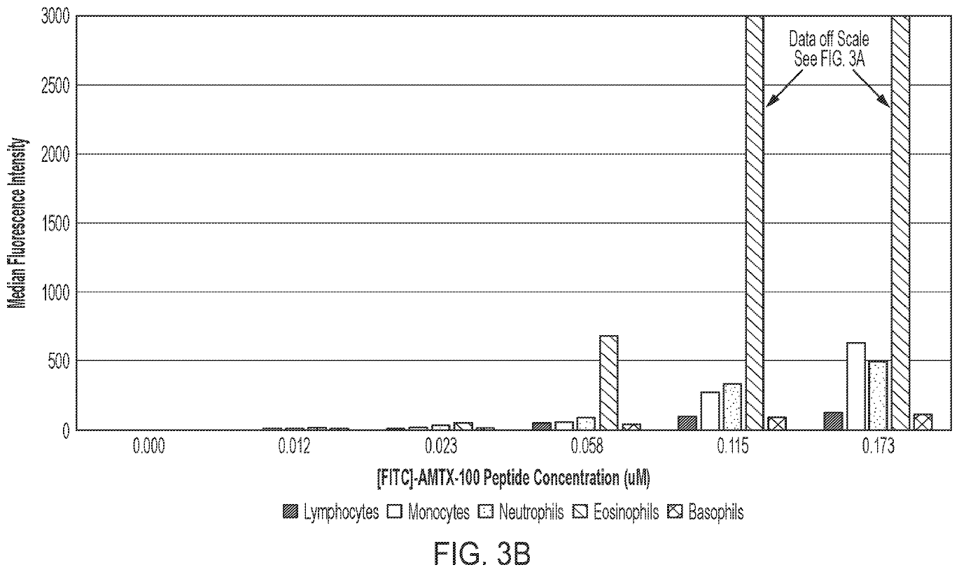

White blood cells from whole mouse blood (n=4 C57BI/6 mice) was pooled and treated with [FITC]-AMTX-100 for 5 min at 37° C. The results demonstrate that [FITC]-AMTX-100 targets eosinophils, neutrophils, and monocytes but not lymphocytes. (FIG. 3A-B).

### Human

Human whole blood (WB) was obtained from multiple normal donors and incubated with [FITC]-AMTX-100 at μg/mL quantities in repeat experiments ex vivo.

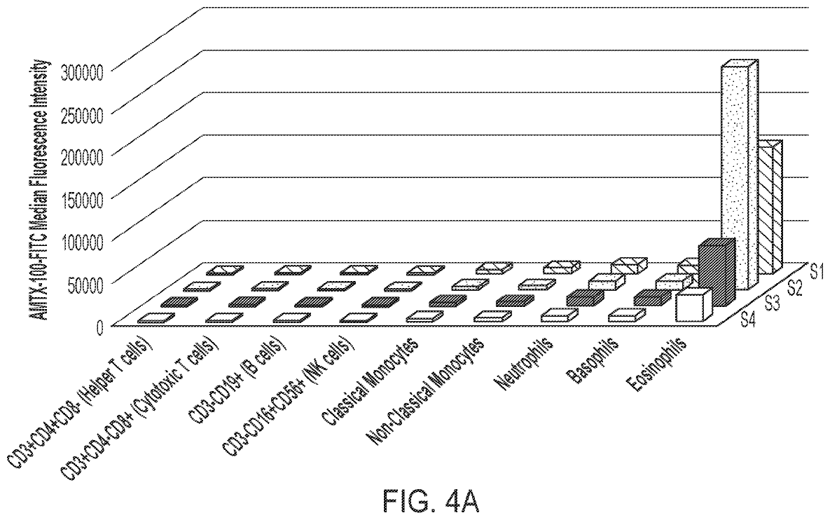

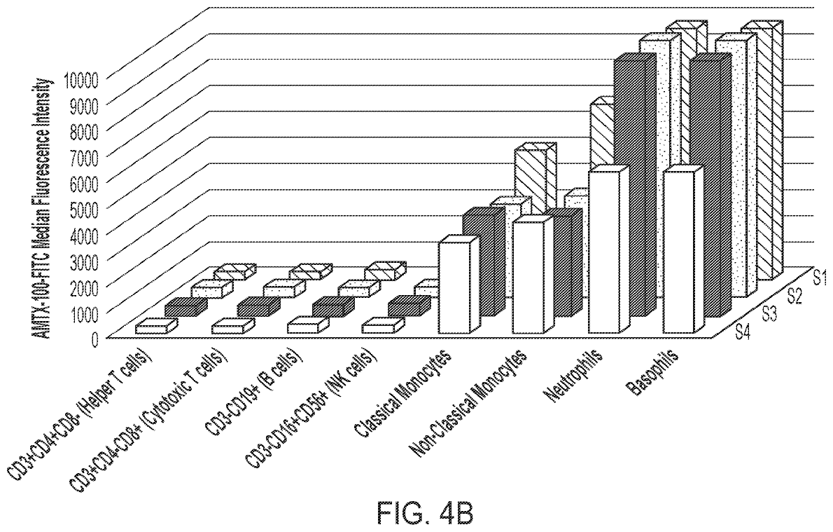

Panels of monoclonal antibodies labeled with fluorescent dyes were used to phenotypically identify [FITC]-AMTX-100 penetrated WB cells including classical monocytes, non-classical monocytes, neutrophils, basophils, eosinophils, and T- and B-lymphocytes and NK cells (FIG. 4A-B). Classical monocytes are characterized by high level expression of the CD14 cell surface receptor (CD14⁺⁺ CD16⁻ monocyte). Non-classical monocytes show low level expression of CD14 and additional co-expression of the CD16 receptor (CD14⁺CD16⁺⁺ monocyte).

RBCs were excluded as they were not penetrated by [FITC]-AMTX-100 and the fluorescence was determined to reside in the WBC leukocyte populations which is consistent with the PK studies finding [³H]-AMTX-100 in the WBC population of WB deduced from various methods of analyses.

The specificity seen in the PK/biodistribution studies in rats that led to the flow cytometry studies with human WB confirm the thesis that AMTX-100 acts through a receptor specifically found on leukocytes (classical and non-classical monocytes, neutrophils, basophils and eosinophils) (FIG. 4A-B).

### Example 4. Staining of RAW 264.7 Cells with Labeled Peptides

FITC-labeled peptides (SEQ ID NOs: 17-20, 22, and 24-26) were constructed to contain the SSHR region (or different versions) with a FITC group attached to a lysine at the amino terminus, an added arginine at the amino terminus, and two lysines added at the carboxy terminus as shown in Table 1.

K (FITC)R-SSHR- KK
Core SSHR:
Human:
(SEQ ID NO: 16)
AAVALLPAVLLALLAP
Mouse:
(SEQ ID NO: 21)
TTGTLLPRVLLALVVA
Rat:
(SEQ ID NO: 23)
TTGTLLPGVLLALVVA

These peptides were used to conduct fluorescent labeling of RAW 264.7 cells (Table 6).

TABLE 6

| | | Activity |
|---|---|---|
| | | (Fluorescence |
| Peptide ID | Sequence | in Cells) |
| SEQ ID NO: 43 | KRAAVALLPKK | − |
| SEQ ID NO: 44 | KRAVLLALLAPKK | + |
| SEQ ID NO: 45 | KRAAVALLAAVLLALLAPKK | + |
| SEQ ID NO: 46 | KRTTGTLLPGVLLALVVAKK | + |
| SEQ ID NO: 47 | KRTTGTLLPGVLLALVVAKK | + |
| SEQ ID NO: 48 | KRTTGTLLPKK | − |
| SEQ ID NO: 49 | KRVLLALVVAKK | + |

Previous studies (U.S. Pat. No. 11,026,992) discloses a peptide comprising a membrane translocating motif (MTM) having the sequence AAVLPVLLAL (SEQ ID NO:27). The results in Table 6 demonstrate that the proline of the prior art sequence is not necessary for binding to RAW264.7 cells as changing the proline to alanine does not alter the binding activity.

### Example 5. Safety and Tolerability of Topically Applied AMTX-100 CF in Adult Patients with Mild to Moderate Atopic Dermatitis

Atopic dermatitis (AD) is a chronic, relapsing, pruritic inflammatory skin disease of unknown origin with eczematous morphology that usually starts in early infancy, but also affects a substantial number of adults. The prevalence of AD among adults in the U.S. is 7.3% and 15%, 15.1%, and 14.5% in 5, 9, and 15 year old children respectively. For the majority of the patients, the disease is resolved by adulthood, but in 10-30% of the affected population it does not. A smaller percentage of the patients develop symptoms later as adults. AD is often associated with a personal or family history of type I allergies with elevated serum immunoglobulin E (IgE) levels, and is associated with pro-inflammatory mediators, such as interleukin 4 (IL-4), IL-13, IL-22, IL-31, interferon gamma (IFN-γ), and thymic stromal lymphopoietin (TSLP), that transduce signals via the Janus kinase-signal transducer and activator of transcription (JAK-STAT) signaling pathway. AD typically presents with pruritus, xerosis, lichenification, and eczematous lesions. Excoriations and crusting are common, and some patients exhibit prurigo nodularis-like lesions. The mainstays of treatment are moisturization (frequent lukewarm baths, and using petrolatum or Aquaphor, etc.), along with topical steroids. For more severe cases, other treatment options such as immunomodulators (ex. tacrolimus and pimecrolimus), biologicals (ex. dupilumab and omalizumab), topical phosphodiesterase-4 (PDE-4) inhibitors (ex. crisaborole), probiotics and phototherapy are also used. Many of these treatment regimens are considered expensive and with various side effects in chronic use. Thus, patients with AD are in need of new treatment options that are efficacious and safe for long-term management of the disease.

Several nonclinical safety studies have been conducted with AMTX-100 CF to evaluate its toxicological profile (Liu X Y, J Biol Chem 275:16774-8, 2000; Liu D L X, J Biol Chem 279:19239-46, 2004; Veach R A, et al. J Biol Chem 279:11425-31, 2004). In this first in human, open-label, dose-escalating, phase I clinical trial, the objectives were to determine the tolerability of topically applied AMTX-100 CF and safety along with evaluating exploratory efficacy end points in adult patients with mild to moderate AD.

### Methods

Study Design. AMTX100-AD-01 study (NCT04313400) was designed as an adaptive phase I/II clinical trial. Only the phase I part of the study is described below. The phase I study was a 6-week, multicenter, open-label, dose-escalation clinical trial evaluating the safety, tolerability and efficacy of AMTX-100 CF in adults with mild to moderate AD. The study included an up to 21-day screening period, a 7-day treatment period, and a 14-day follow-up period. A central Institutional Review Board (IRB) approved the study protocol, informed consent form, study sites and recruitment materials before patient enrollment. This clinical trial was conducted according to the International Council for Harmonization (ICH) guidelines, applicable regulations, and the Declaration of Helsinki. Patients were provided written informed consents before screening and initiation of any study related procedures.

Clinical Trial Procedures. Twenty-six (26) patients were sequentially enrolled in five (5) cohort dose levels (per the body surface area [BSA] affected with AD) by escalating applications of AMTX-100 CF 1.1%. The enrolled patients were assigned to receive AMTX-100 CF twice daily, for 7 days starting at the baseline visit.

The applied amount of AMTX-100 CF depended on the percentage of the BSA affected with AD as assessed by the investigator, and per the instructions in the protocol (approximately 1 g cream per 400 cm²). First dose of the study treatment was administered topically at the study clinic during the baseline visit by trained site staff. The subjects were trained on administration techniques, including appropriate amount for topical use, how to measure assigned dose and how to record study treatment compliance in patient diary. All other doses were self-administered by the subjects at home. The subjects had to apply the AMTX-100 CF topical cream to all AD lesions (excluding the scalp, face, eyes, eyelids, neck, hands, palms, feet, groin, genitals or in the axillae) for 7 consecutive days (14 applications in total), regardless of whether the lesions became clinically clear during the 7-day treatment period. Rescue therapy, defined as any topical or systemic immunomodulatory treatment initiated for AD, could be given at any time per investigators discretion. In case of administering rescue therapy, the subject would be considered treatment failure for analyses purposes.

The decision for cohort (dose level) escalation (to enroll patients with higher BSA involved with AD) was made by a Safety Assessment Committee (SAC) after reviewing the safety data for the previous cohort. In case any dose limiting toxicities (DLTs) were observed, the study Data Safety Monitoring Committee (DSMC) had to conduct an independent review of the data to make a final recommendation for dose escalation to the next cohort.

Safety Parameters. The primary safety outcome measure was determining Maximum Tolerable Dose (MTD) of AMTX-100 CF (1.1%) (as per the maximum percentage of BSA treated) by evaluation of the DLTs. Other Safety outcome measures were treatment-emergent adverse events (TEAEs), clinically significant changes and shift in laboratory measurements, and vital signs in all patients who received 1 or more doses of study drug through the follow-up period. TEAEs were defined as any adverse event (AE) that began or worsened in severity after initiation of AMTX-100 CF use. All AEs presented were treatment-emergent, unless otherwise noted.

Efficacy parameters. Efficacy outcome measures included change from baseline in the treated BSA percentage and change from baseline in vIGA-AD™ at days 7 (end of treatment) and 21 (end of follow-up).

Statistical Analysis. Statistical analysis was conducted on the Safety population defined as any subject receiving the study treatment. Statistical analyses were performed using SAS® for Windows, version 9.4. Descriptive statistics (n, mean, standard deviation, median, minimum and maximum) were calculated for continuous variables. Frequencies and percentages were presented for categorical variables.

### Results

A total of 26 subjects were enrolled in the Part 1 (Phase I) of the study to receive AMTX-100 CF 1.1%. All subjects finished the entire course of treatment and follow-up with five (5) subjects in each of the Cohorts 1, 2, and 3, four (4) subjects in Cohort 4, and seven (7) subjects in Cohort 5 (Table 7). Baseline demographics and disease characteristics of the subjects are presented in Table 8.

TABLE 7
Affected BSA areas per cohort

| | Treated Area of the BSA | | No. of Subjects Enrolled | | |
|---|---|---|---|---|---|
| | (percentage) | | Completed | Withdrawn | Total |
| Cohort 1 | 3%-6% | BSA | 5 | 0 | 5 |
| Cohort 2 | 6%-12% | BSA | 5 | 0 | 5 |
| Cohort 3 | 12%-24% | BSA | 5 | 0 | 5 |
| Cohort 4 | 24%-48% | BSA | 4 | 0 | 4 |
| Cohort 5 | 48%-70% | BSA | 7 | 0 | 7 |
| Subtotal | | | 26 | 0 | 26 |

Among the 26 enrolled subjects, 14 (54%) were male. The mean age was 48.7 years with a range between 20 to 75 years. Safety was assessed by evaluating treatment-emergent adverse events (TEAEs) in all 26 subjects. AEs were classified by system organ class (SOC) and preferred term (PT) according to the MedDRA dictionary (version 23.1).

TABLE 8
Baseline Demographic and Disease characteristics

| | Subject Numbers (%) | |
|---|---|---|
| Characteristics | N = 26 | |
| Sex | | |
| Male | 14 | (53.8) |
| Female | 12 | (46.2) |
| Age, Mean (SD) [Range] | 48.7 (18.22), [20-75] | |
| BMI, Mean (SD) | 27 | (5.8) |
| BSA, Mean (SD) | 1.9 | (0.24) |
| Calculated Treatable BSA Percentage, Mean | 24.0 (19.50) [3-62] | |
| (SD) [Range] % | | |
| VIGA-AD ™ Score | | |
| 2- Mild | 11 | (42.3) |
| 3-Moderate | 15 | (57.7) |

Abbreviations:
BMI: body mass index;
BSA: body surface area;
vIGA-AD ™: validated Investigator's Global Assessment for Atopic Dermatitis

### Safety Outcomes

Eight out of 26 subjects experienced TEAEs. The most frequent TEAE term reported was headache (mostly mild), experienced by 5 subjects. Three AEs with moderate severity (viral upper respiratory and urinary tract infections, headache) were reported in three subjects. No significant changes and shifts in laboratory measurements and vital signs were detected for any of the enrolled patients during the study.

Study treatment application site reactions were defined as symptoms potentially associated with applying the topical study treatment, including dryness, erythema/skin irritation, burning/stinging, erosion/ulceration, edema/swelling, itching, pain on treated area, crusting, vesiculation/pustulation, flaking/scaling and bleeding which were assessed at the baseline (pre-dose, post-dose), end of the treatment, and follow-up visits. Symptoms similar to application site reactions existed at the AD lesions (due to the nature of AD lesions) prior to the first application of the study drug, however, they mostly improved or resolved during the study after study treatment administration. Post-baseline study treatment application site reactions (burning/stinging, dryness and itching) occurred in 4 of 26 (15.5%) subjects. In all of these subjects the reactions were mild to moderate and in three of the subjects improved during the study. There was just one subject who experienced mild itching at the end of treatment visit, which continued through follow-up.

None of the TEAEs were considered to be study treatment related. All AEs were resolved. No AEs led to study drug discontinuation, and no SAE or Death was reported in this study.

The decision for cohort (dose level) escalation to the higher dose level (based on higher BSA to be treated with greater amounts of AMTX-100 CF 1.1%) was based on lack of any Dose Limiting Toxicities (DLTs). No DLTs occurred in any cohorts, thus the dose escalation continued safely through the last cohort (cohort 5). Maximum daily amount of AMTX-100 CF assigned to subjects was 60 g/day for the highest cohort (with 48-70% of treatable BSA).

### Efficacy Results

Compared to baseline, the mean percentage of BSA affected by AD decreased at the Day 7 visit (end of treatment, for all cohorts) and follow-up visit (for cohorts 4 and 5 only) by 37.5% and 37.9% respectively (Table 4). Per cohort reductions at day 7 compared to baseline were 46.7% (cohort 1), 44.3% (cohort 2), 43.3% (cohort 3), 38.1% (cohort 4) and 21.7% (cohort 5).

The affected BSA with AD was not evaluated for cohorts 1 to 3 at the follow-up visit. Cohorts 4 and 5 reductions at the follow-up visit compared to baseline were 42.1% (cohort 4) and 35.1% (cohort 5).

Across the study cohorts, Validated Investigator Global Assessment for Atopic Dermatitis (vIGA-AD™) scores improved after the AMTX-100 CF intervention. At the baseline, 11 (42.3%) and 15 (57.7%) subjects had grade 2 (mild) and grade 3 (moderate) vIGA scores accordingly.

By day 7 (end of treatment), 3 subjects (11.5%) had grade 0 (clear) score, 10 subjects (38.5%) had grade 1 (almost clear) score, 5 subjects (19.2%) had grade 2 (mild) score, and 8 subjects (30.8%) had grade 3 (moderate) score.

At day 21 (follow-up) visit, 4 subjects (15.4%) had achieved grade 0 (clear) score, 10 subjects (38.5%) had grade 1 (almost clear) score, 7 subjects (26.9%) had grade 2 (mild) score and 5 subjects (19.2%) had grade 3 (moderate) score (Table 9).

TABLE 9
vIGA-AD score changes during the study

| | | Number/Percentage of Subjects | | | | | | | | | | | |
|---|---|---|---|---|---|---|---|---|---|---|---|---|---|
| | | Cohort 1 | | Cohort 2 | | Cohort 3 | | Cohort 4 | | Cohort 5 | | Total | |
| Visit | vIGA Score | N = 5 | | N = 5 | | N = 5 | | N = 4 | | N = 7 | | N = 26 | |
| Baseline | 2-Mild | 5 | (100.0) | 4 | (80.0) | 1 | (20.0) | 1 | (25.0) | 0 | (0.0) | 11 | (42.3) |
| (n, %) | 3-Moderate | 0 | (0.0) | 1 | (20.0) | 4 | (80.0) | 3 | (75.0) | 7 | (100.0) | 15 | (57.7) |
| End of | 0-Clear | 1 | (20.0) | 1 | (20.0) | 1 | (20.0) | 0 | (0.0) | 0 | (0.0) | 3 | (11.5) |
| Treatment | 1-Almost Clear | 3 | (60.0) | 3 | (60.0) | 2 | (40.0) | 1 | (25.0) | 1 | (14.3) | 10 | (38.5) |
| (n, %) | 2-Mild | 1 | (20.0) | 0 | (0.0) | 0 | (0.0) | 2 | (50.0) | 2 | (28.6) | 5 | (19.2) |
| | 3-Moderate | 0 | (0.0) | 1 | (20.0) | 2 | (40.0) | 1 | (25.0) | 4 | (57.1) | 8 | (30.8) |
| Follow-Up | 0-Clear | 3 | (60.0) | 0 | (0.0) | 1 | (20.0) | 0 | (0.0) | 0 | (0.0) | 4 | (15.4) |
| (n, %) | 1-Almost Clear | 0 | (0.0) | 4 | (80.0) | 2 | (40.0) | 1 | (25.0) | 3 | (42.9) | 10 | (38.5) |
| | 2-Mild | 2 | (40.0) | 1 | (20.0) | 2 | (40.0) | 2 | (50.0) | 0 | (0.0) | 7 | (26.9) |
| | 3-Moderate | 0 | (0.0) | 0 | (0.0) | 0 | (0.0) | 1 | (25.0) | 4 | (57.1) | 5 | (19.2) |

### CONCLUSION

Currently approved topical therapies, corticosteroids and calcineurin inhibitors have been shown to be efficacious in improvement of mild to moderate AD, but long-term use of these medications can be associated with safety risks such as striae, increase risk of adrenal suppression, and slow linear growth with long term use of glucocorticoids.

Disclosed herein is a new approach to treating AD that involves the inhibition of key mediators of inflammation. The competitive binding of AMTX-100 to Imp α/β complex or to Imp β leads to a reduction in nuclear import of SRTFs in inflammation and ChREBP and SHREBP in metabolic syndrome, thus leading to a reduction in pro-inflammatory cytokine/chemokine production or lipid metabolic products, respectively. AMTX-100 has excellent potential to suppress inflammatory responses and reduce related symptoms.

During this study, AMTX-100 CF 1.1% topical cream was generally well tolerated. The most frequently reported TEAEs were mild to moderate headache that occurred in 5 of 26 subjects and were not associated with any specific cohort. All were resolved after medication administration and were considered to be unrelated to the study treatment. There were no serious adverse events (SAEs). No adverse events that led to study treatment discontinuation and no deaths occurred. No DLTs occurred at any cohorts of the study. The study treatment was administered at up to 60 gr/day to the subjects in the cohort 5 with highest amount of BSA involved (48-70%) with AD.

In terms of efficacy, AMTX-100 CF 1.1% topical cream improved disease severity and intensity and also reduced the areas affected by AD lesions in all cohorts.

### Example 6. Conjugation of Methotrexate to AMTX-100

Methotrexate was conjugated to AMTX-100 as depicted in FIG. 7.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A leukocyte-targeting molecule, comprising a peptide having the formula:
(SEQ ID NO: 10)
X¹X²AAX³AX⁴X⁵X¹⁷AX⁶X⁷X⁸AX⁹X¹⁰A(P)ₙ¹¹X¹²(X¹³)ₙ
or a pharmaceutically acceptable salt thereof;
wherein
X¹, X², X¹¹, and X¹² are each, independently, lysine, arginine, or ornithine;
X³, X⁴, X⁵, X⁶, X⁷, X⁸, X⁹, and X¹⁰ are each, independently, valine, leucine, isoleucine, or norleucine;
X¹³ is tyrosine;
X¹⁷ is proline or alanine; and
n is 0 or 1.

2. The leukocyte-targeting molecule of claim 1, wherein X³ and X⁶ are valine.

3. The leukocyte-targeting molecule of claim 1, wherein X⁴, X⁵, X⁷, X⁸, X⁹, and X¹⁰ are each, independently, leucine, isoleucine, or norleucine.

4. The leukocyte-targeting molecule of claim 1, wherein the peptide has a formula:
(SEQ ID NO: 11)
X¹X²AAVALLPAVLLALLA(P)ₙX¹¹X¹²(X¹³)ₙ
or a pharmaceutically acceptable salt thereof.

5. The leukocyte-targeting molecule of claim 1, wherein the peptide comprises at least one ornithine, isoleucine, or norleucine.

6. The leukocyte-targeting molecule of claim 1, wherein the peptide has a formula selected from KKAAVALLPAVLLALLAKK (SEQ ID NO:5), RRAAVALLPAVLLALLARR (SEQ ID NO:6), RRAAVALLPAVLLALLARK (SEQ ID NO:7), RKAAVALLPAVLLALLARKY (SEQ ID NO:8), AAVALLPAVLLALLAPCVQRKRQKLMPC (SEQ ID NO: 9), AAVALLPAVLLALLAPVQRKRQKLMP (SEQ ID NO:13), KKAAVALLPAVLLALLAPKK (SEQ ID NO:39), RRAAVALLPAVLLALLAPRR (SEQ ID NO:40), RRAAVALLPAVLLALLAPRK (SEQ ID NO:41), or RKAAVALLPAVLLALLAPRKY (SEQ ID NO: 42).

7. The leukocyte-targeting molecule of claim 1, further comprising the amino acid sequence of CVQRKRQKLMPC (SEQ ID NO:38) at the C-terminus.

8. The leukocyte-targeting molecule of claim 1, wherein the peptide has a formula:
(SEQ ID NO: 12)
X¹X²AAX³AX⁴X⁵X¹⁷AX⁶X⁷X⁸AX⁹X¹⁰A(P)ₙX¹¹X¹²(X¹³)ₙCVQRKRQK
LMPC
or a pharmaceutically acceptable salt thereof.

9. The leukocyte-targeting molecule of claim 7, wherein the peptide is cyclized at the cysteine residues.

10. The leukocyte-targeting molecule of claim 1, wherein the peptide is linear.

11. The leukocyte-targeting molecule of claim 1, wherein the peptide further comprises one or two C-terminal lysine.

12. The leukocyte-targeting molecule of claim 1, wherein the peptide further comprises a N-terminal lysine.

13. The leukocyte-targeting molecule of claim 1, wherein the peptide further comprises an arginine residue disposed between the N-terminal lysine and X¹.

14. The leukocyte-targeting molecule of claim 1, wherein the compound comprises at least one radioisotope of iodine.

15. The leukocyte-targeting molecule of claim 14, wherein the at least one radioisotope of iodine is selected, independently, from ¹²³I, ¹²⁴I, ¹²⁵I, or ¹³¹I.

16. The leukocyte-targeting molecule of claim 1, wherein the peptide comprises at least one D-amino acid.

17. The leukocyte-targeting molecule of claim 1, wherein the leukocyte is an eosinophil, a basophil, a neutrophil, or a monocyte.

18-19. (canceled)

20. A method of delivering an active pharmaceutical agent to a leukocyte, a CD34⁺ stem cell, or both, in a subject in need thereof, comprising administering the leukocyte-targeting molecule of claim 1 to the subject.

21. The method of claim 20, wherein the leukocyte is an eosinophil, a basophil, a neutrophil, or a monocyte.

22. A method of treating a disease in a subject, comprising administering a therapeutically effective amount of the leukocyte-targeting molecule of claim 1 to the subject.

23. The method of claim 22, wherein the disease is an inflammatory disease, an autoimmune disease, a skin disorder, a viral disease, atherosclerosis, non-alcoholic steatohepatitis, hypercholesteremia, age-related macular degeneration, diabetic retinopathy, conjunctivitis, uveitis, or chronic sinusitis.

24.-39. (canceled)


SEQUENCE LISTING (52 sequences)

SEQ ID NO: 1 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 2 - protein, 13 aa, synthetic construct
  VARIANT 8..9: note = X = Leu, Ile, or Nle
  VARIANT 11..12: note = X= Leu, Ile, or Nle
AAVAPAVXXAXXA

SEQ ID NO: 3 - protein, 16 aa, synthetic construct
AAVALLPAVLLALLAP

SEQ ID NO: 4 - protein, 19 aa, synthetic construct
  VARIANT 1..2: note = X=Lys, Arg, or Orn
  VARIANT 18..19: note = X= Lys, Arg, or Orn
XXAAVALLPAVLLALLAXX

SEQ ID NO: 5 - protein, 19 aa, synthetic construct
KKAAVALLPAVLLALLAKK

SEQ ID NO: 6 - protein, 19 aa, synthetic construct
RRAAVALLPAVLLALLARR

SEQ ID NO: 7 - protein, 19 aa, synthetic construct
RRAAVALLPAVLLALLARK

SEQ ID NO: 8 - protein, 20 aa, synthetic construct
RKAAVALLPAVLLALLARKY

SEQ ID NO: 9 - protein, 28 aa, synthetic construct
AAVALLPAVLLALLAPCVQRKRQKLMPC

SEQ ID NO: 10 - protein, 21 aa, synthetic construct
  VARIANT 1..2: note = X=Lys, Arg, Orn
  VARIANT 5: note = X=Val, Leu, Ile, or Nle
  VARIANT 7..8: note = X=Val, Leu, Ile, or Nle
  VARIANT 9: note = X=Pro or Ala
  VARIANT 11..13: note = X=Val, Leu, Ile, or Nle
  VARIANT 15..16: note = X=Val, Leu, Ile, or Nle
  VARIANT 19..20: note = X= Lys, Arg, or Orn
  VARIANT 18: note = Pro may be deleted
  VARIANT 21: note = X=Tyr or null
XXAAXAXXXAXXXAXXAPXXX

SEQ ID NO: 11 - protein, 21 aa, synthetic construct
  VARIANT 1..2: note = X=Lys, Arg, or Orn
  VARIANT 19..20: note = X=Lys, Arg, or Orn
  VARIANT 21: note = X=Tyr or null
XXAAVALLPAVLLALLAPXXX

SEQ ID NO: 12 - protein, 33 aa, synthetic construct
  VARIANT 1..2: note = X=Lys, Arg, or Orn
  VARIANT 5: note = X=Val, Leu, Ile, or Nle
  VARIANT 7..8: note = X=Val, Leu, Ile, or Nle
  VARIANT 9: note = X=Pro or Ala
  VARIANT 11..13: note = X=Val, Leu, Ile, or Nle
  VARIANT 15: note = X=Arg
  VARIANT 16: note = X=Leu, Ile, or Nle
  VARIANT 19..20: note = X=Lys, Arg, or Orn
  VARIANT 21: note = X=Tyr or null
XXAAXAXXXAXXXAXXAPXXXCVQRKRQKLMPC

SEQ ID NO: 13 - protein, 26 aa, synthetic construct
AAVALLPAVLLALLAPVQRKRQKLMP

SEQ ID NO: 14 - protein, 28 aa, synthetic construct
AAVALLPAVLLALLAPCYVQRKRQKLMP

SEQ ID NO: 15 - protein, 14 aa, synthetic construct
  VARIANT 8..9: note = X=Leu, Ile, or Nle
  VARIANT 11..12: note = X=Leu, Ile, or Nle
AAVAPAVXXAXXAP

SEQ ID NO: 16 - protein, 16 aa, synthetic construct
AAVALLPAVLLALLAP

SEQ ID NO: 17 - protein, 10 aa, synthetic construct
  VARIANT 1: note = X = Lys or null
  VARIANT 2: note = X=Arg or null
  VARIANT 10: note = X=Lys-Lys or null
XXAAVALLPX

SEQ ID NO: 18 - protein, 12 aa, synthetic construct
  VARIANT 1: note = X=Lys or null
  VARIANT 2: note = X=Arg or null
  VARIANT 12: note = X=Lys-Lys or null
XXAVLLALLAPX

SEQ ID NO: 19 - protein, 28 aa, synthetic construct
AAVALLPAVLLAVLAPCVQRDEQKLMPC

SEQ ID NO: 20 - protein, 19 aa, synthetic construct
  VARIANT 1: note = X=Lys or null
  VARIANT 2: note = X=Arg or null
  VARIANT 19: note = X=Lys-Lys or null
XXAAVALLAAVLLALLAPX

SEQ ID NO: 21 - protein, 16 aa, synthetic construct
TTGTLLPGVLLALVVA

SEQ ID NO: 22 - protein, 19 aa, synthetic construct
  VARIANT 1: note = X=Lys or null
  VARIANT 2: note = X=Arg or null
  VARIANT 19: note = X=Lys-Lys or null
XXTTGTLLPGVLLALVVAX

SEQ ID NO: 23 - protein, 16 aa, synthetic construct
TTGTLLPRVLLALVVA

SEQ ID NO: 24 - protein, 19 aa, synthetic construct
  VARIANT 1: note = X=Lys or null
  VARIANT 2: note = X=Arg or null
  VARIANT 19: note = X=Lys-Lys or null
XXTTGTLLPGVLLALVVAX

SEQ ID NO: 25 - protein, 10 aa, synthetic construct
  VARIANT 1: note = X=Lys or null
  VARIANT 2: note = X=Arg or null
  VARIANT 10: note = X=Lys-Lys or null
XXTTGTLLPX

SEQ ID NO: 26 - protein, 11 aa, synthetic construct
  VARIANT 1: note = X=Lys or null
  VARIANT 2: note = X=Arg or null
  VARIANT 11: note = X=Lys-Lys or null
XXVLLALVVAX

SEQ ID NO: 27 - protein, 10 aa, synthetic construct
AAVLPVLLAL

SEQ ID NO: 28 - protein, 19 aa, synthetic construct
  VARIANT 19: note = X=Lys-Lys or null
RAAVALLPAVLLALLAPYX

SEQ ID NO: 29 - protein, 19 aa, synthetic construct
  VARIANT 19: note = X=Lys-Lys or null
RAAVALLPAVLLAVLAPYX

SEQ ID NO: 30 - protein, 32 aa, synthetic construct
  VARIANT 1: note = X=Lys or null
  VARIANT 2: note = X=Arg or null
  VARIANT 31: note = X=Lys or null
  VARIANT 32: note = Tyr-Tyr or null
XXAAVALLPAVLLALLAPDVRKRQDLEQKMXY

SEQ ID NO: 31 - protein, 19 aa, synthetic construct
  VARIANT 1: note = X=Lys or null
  VARIANT 2: note = X=Arg or null
  VARIANT 19: note = X=Lys-Lys or null
XXAAVALLPAVLLALLAPX

SEQ ID NO: 32 - protein, 19 aa, synthetic construct
  VARIANT 1: note = X=Lys or null
  VARIANT 2: note = X=Arg or null
  VARIANT 19: note = X=Lys-Lys or null
XXAAVALLAAVLLALLAPX

SEQ ID NO: 33 - protein, 19 aa, synthetic construct
  VARIANT 1: note = X=Lys
  VARIANT 2: note = X=Arg or null
  VARIANT 19: note = X=Lys-Lys or null
XXTTGTLLPGVLLALVVAX

SEQ ID NO: 34 - protein, 19 aa, synthetic construct
  VARIANT 1: note = X=Lys or null
  VARIANT 2: note = X=Arg or null
  VARIANT 19: note = X=Lys-Lys or null
XXTTGTLLPRVLLALVVAX

SEQ ID NO: 35 - protein, 10 aa, synthetic construct
  VARIANT 1: note = X=Lys or null
  VARIANT 2: note = X=Arg or null
  VARIANT 10: note = X=Lys-Lys or null
XXTTGTLLPX

SEQ ID NO: 36 - protein, 12 aa, synthetic construct
  VARIANT 1: note = X=Lys or null
  VARIANT 2: note = X=Arg or null
  VARIANT 12: note = X=Lys-Lys or null
XXGVLLALVVAX

SEQ ID NO: 37 - protein, 29 aa, synthetic construct
  VARIANT 1: note = X=Lys or null
  VARIANT 2: note = X=Arg or null
XXAAVALLPAVLLALLAPCVQKRQKLMPC

SEQ ID NO: 38 - protein, 12 aa, synthetic construct
CVQRKRQKLMPC

SEQ ID NO: 39 - protein, 20 aa, synthetic construct
KKAAVALLPAVLLALLAPKK

SEQ ID NO: 40 - protein, 20 aa, synthetic construct
RRAAVALLPAVLLALLAPRR

SEQ ID NO: 41 - protein, 20 aa, synthetic construct
RRAAVALLPAVLLALLAPRK

SEQ ID NO: 42 - protein, 21 aa, synthetic construct
RKAAVALLPAVLLALLAPRKY

SEQ ID NO: 43 - protein, 11 aa, synthetic construct
KRAAVALLPKK

SEQ ID NO: 44 - protein, 13 aa, synthetic construct
KRAVLLALLAPKK

SEQ ID NO: 45 - protein, 20 aa, synthetic construct
KRAAVALLAAVLLALLAPKK

SEQ ID NO: 46 - protein, 20 aa, synthetic construct
KRTTGTLLPGVLLALVVAKK

SEQ ID NO: 47 - protein, 20 aa, synthetic construct
KRTTGTLLPGVLLALVVAKK

SEQ ID NO: 48 - protein, 11 aa, synthetic construct
KRTTGTLLPKK

SEQ ID NO: 49 - protein, 12 aa, synthetic construct
KRVLLALVVAKK

SEQ ID NO: 50 - protein, 11 aa, synthetic construct
KRAAVALLPKK

SEQ ID NO: 51 - protein, 28 aa, synthetic construct
AAVALLPAVLLAVLAPCVQRKRQKLMPC

SEQ ID NO: 52 - protein, 28 aa, synthetic construct
AAVALLPAVLLALLAPCVQRDEQKLMPC